(19) United States
(12) Patent Application Publication
(10) Pub. No.: US 2026/0325775 A1
(43) Pub. Date: Oct. 22, 2026

(54) ENDOTRACHEAL TUBE

(71) Applicant: KING SAUD UNIVERSITY, Riyadh (SA)

(72) Inventors: KAWTHAR MOHAMMED ALSALEH, Riyadh (SA); ADNAN MOHAMMED ALMOUSAWI, Riyadh (SA)

(21) Appl. No.: 19/180,487

(22) Filed: Apr. 16, 2025

(51) Int. Cl. A61M 16/04

(52) U.S. Cl. CPC A61M 16/0402; A61M 16/0472; A61M 2210/1032

(57) ABSTRACT

An endotracheal tube comprising a tubular wall having an open proximal end, an opposing open distal end, and a distal wall portion spaced from the first end and extending to the second end; a plurality of perforations defined through an anterior wall segment of the distal wall portion; a metal mesh formed along the anterior wall segment, the metal mesh being in communication with an electronic indicator; and a metal plate formed along a posterior wall segment of the distal wall portion, the metal plate being in communication with the electronic indicator.

### BACKGROUND

### 1. Field

The disclosure of the present patent application relates to a device for assisting in tracheostomy and, particularly, to an endotracheal tube set that can be administered to a patient without requiring endoscopy.

### 2. Description of the Related Art

Tracheostomy creation is a routine procedure in critical care settings, performed for a wide range of medical conditions that require airway management. Despite its widespread use in critical care, current techniques for tracheostomy creation present notable limitations that must be addressed. These challenges include risks such as tracheal wall rupture, bleeding, and complications in airway management during the procedure. Enhancing the procedure with improved tools and methods is essential to minimize these risks and optimize patient outcomes.

Nowadays, it is estimated that 14 out of 100 intubated patients inside critical care units (ICUs) will eventually need a tracheostomy tube during their hospital admission. Currently, there are two main methods for this procedure

The first approach is surgical tracheostomy, typically performed in an operating room. This method involves making an incision in the neck and directly accessing the trachea to create an airway. The procedure is performed by a surgical team, and requires booking of the operation room, in advance. In addition, the procedure requires the presence of qualified surgeons to perform the procedures, an anesthetist, and a scrubbing and circulating nurse. Furthermore, the risk of bleeding, surgical site infection and esophageal perforation is relatively higher with this method.

The second approach is percutaneous tracheostomy, which is considered to be a less invasive, bedside procedure often performed in ICUs. It involves inserting a needle and guidewire into the trachea, followed by dilation to create a stoma for the tracheostomy tube. The conventional practice of the percutaneous tracheostomy is to palpate the landmarks (thyroid cartilage, cricoid cartilage, tracheal rings) to confirm the area between the 2nd and 3rd tracheal rings while also using a bronchoscopy for guidance during the needle puncture. In the conventional practice, the endotracheal tube is removed prior to starting the percutaneous tracheostomy procedure to allow for insertion of the needle, followed by the guidewire, first-stage dilator, guidewire catheter protector, second-stage dilator, then tracheostomy and visualization using bronchoscopy. Though less invasive and quicker to perform, this method can be technically challenging, especially in patients with difficult anatomy, and carries the risk of complications such as damage to adjacent structures. The procedure necessitates the presence of at least two physicians, one performing the procedure, and the other helping with the visualization using bronchoscopy. In addition to the requirement for two physicians, one with expertise in bronchoscopy, a major limitation of this procedure is the necessity of bronchoscopy, which significantly increases the cost.

Thus, conventional practice requires a dedicated medical team and advanced equipment. Moreover, the high risk of complications, such as insertion errors and esophageal perforation, highlights the pressing need for solutions and the development of more innovative devices to enhance safety and improve outcomes.

Thus, an endotracheal tube solving the aforementioned problems is desired.

### SUMMARY

In certain embodiments, the present subject matter relates to an endotracheal tube that facilitates safe insertion of the tracheostomy tube into the trachea of a patient. The endotracheal tube includes a tubular wall having an open proximal end, an opposing open distal end, and a distal wall portion spaced from the first end and extending to the second end; a plurality of perforations defined through an anterior wall segment of the distal wall portion; a metal mesh formed along the anterior wall segment, the metal mesh being in communication with an electronic indicator; and a metal plate formed along a posterior wall segment of the distal wall portion, the metal plate being in communication with the electronic indicator.

According to an embodiment, an endotracheal tube includes a tubular wall having an open proximal end, an opposing open distal end, and a distal wall portion spaced from the first end and extending to the second end; a plurality of perforations defined through an anterior wall segment of the distal wall portion; a metal mesh formed along the anterior wall segment, the metal mesh being in communication with an electronic indicator; and a metal plate formed along a posterior wall segment of the distal wall portion, the metal plate being in communication with the electronic indicator and including first and second parallel, opposing sides, third and fourth parallel, opposing sides, and a fifth slanted side extending between and connecting the second and third sides.

According to an embodiment, an endotracheal tube includes a tubular wall having an open proximal end, an opposing open distal end, and a distal wall portion spaced from the first end and extending to the second end; a plurality of perforations defined through an anterior wall segment of the distal wall portion; a metal mesh formed along the anterior wall segment, the metal mesh being in communication with an electronic indicator; and a metal plate formed along a posterior wall segment of the distal wall portion, the metal plate being in communication with the electronic indicator, wherein the metal mesh is in communication with the electronic indicator through a first electric circuit and the metal plate is in communication with the electronic indicator through a second electric circuit.

These and other features of the present subject matter will become readily apparent upon further review of the following specification.

### BRIEF DESCRIPTION OF THE DRAWINGS

FIG. 1 is a perspective view of the endotracheal tube according to the present teachings and a monitor in communication with the endotracheal tube.

FIG. 2A is an exploded, perspective view of the distal portion of the endotracheal tube according to the present teachings.

FIG. 2B is an exploded, cutaway view of the distal portion of the endotracheal tube according to the present teachings.

FIG. 2C is an exploded view of the inner surface of the anterior segment of the endotracheal tube according to the present teachings.

FIG. 3 is an exploded view of the inner surface of the anterior segment of the endotracheal tube according to the present teachings.

FIG. 4A shows the metal plate in the appropriate position for proper placement of the tracheostomy tube.

FIG. 4B shows the metal plate angled such that the slanted side is not visible, indicating that the endotracheal tube is improperly rotated.

FIG. 4C shows the metal plate with the slanted side at the top left of the metal plate, indicating that the endotracheal tube is in a flipped orientation.

FIG. 5 shows a plan view of the distal end of the endotracheal tube, with the metal plate disposed within the posterior wall segment.

FIGS. 6A-6G depict an exemplary method for placement of the tracheostomy tube.

### DETAILED DESCRIPTION OF THE PREFERRED EMBODIMENTS

The following definitions are provided for the purpose of understanding the present subject matter and for construing the appended patent claims.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms "include," "includes", "including," "have," "has," or "having" should be generally understood as open-ended and non-limiting unless specifically stated otherwise.

The use of the singular herein includes the plural (and vice versa) unless specifically stated otherwise. In addition, where the use of the term "about" is before a quantitative value, the present teachings also include the specific quantitative value itself, unless specifically stated otherwise. As used herein, the term "about" refers to a ±10% variation from the nominal value unless otherwise indicated or inferred.

The term "optional" or "optionally" means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In one embodiment, the present subject matter relates to an endotracheal tube, designated 10 in the drawings. The endotracheal tube 10 facilitates safe insertion of a tracheostomy tube into the trachea of a patient, without the aid of bronchoscopy, by receiving the tracheostomy tube at the desired position of the trachea (between the 2^{nd} and 3^{rd} tracheal rings). Once the tracheostomy tube is properly positioned, the endotracheal tube 10 can be removed from the patient.

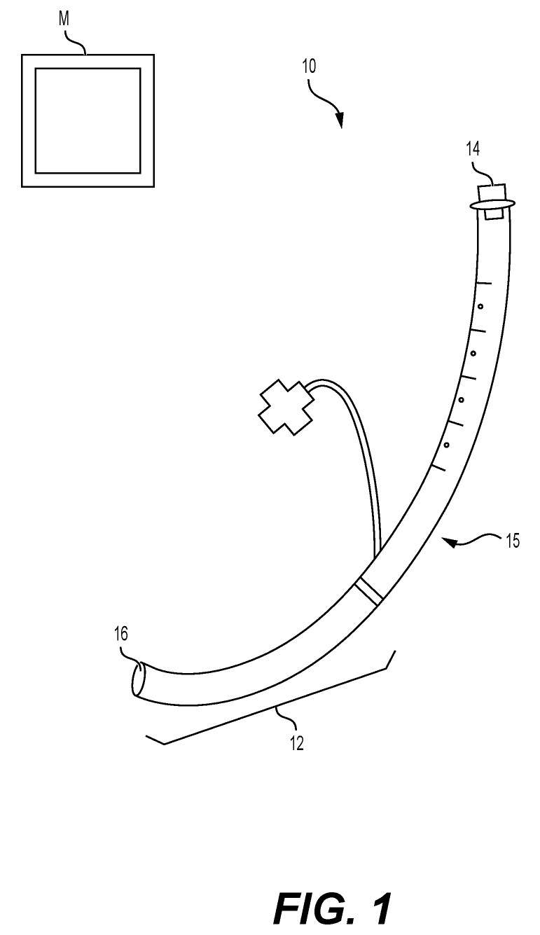

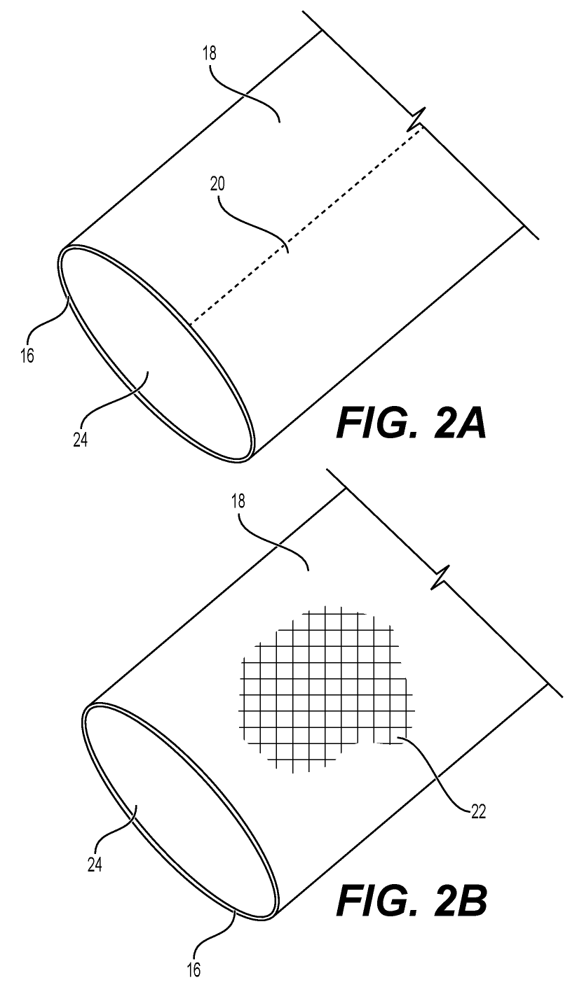

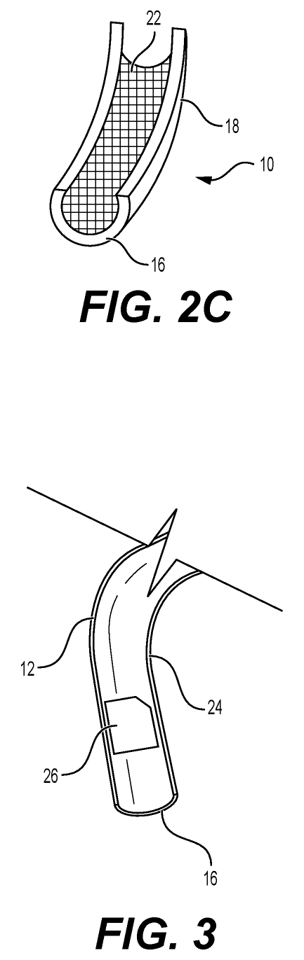

The endotracheal tube 10 can include an open proximal end 14, an open distal end 16, and a tubular wall 15 extending between the proximal and distal ends, 14 and 16 (FIG. 1). As shown in FIGS. 2A-2C, a distal wall portion 12 of the tubular wall 15 can be spaced from the first end 14 and extend to the second end 16. A plurality of perforations 20 can be defined through an anterior wall segment 18 the distal wall portion 12. The anterior wall segment 18 can further include a metal mesh 22, e.g., along an inner surface thereof. A posterior wall segment 24 of the distal wall portion 12 (FIG. 3), opposite the anterior wall segment 18, can include a metal plate 26. The metal mesh 22 can be in communication with an electronic indicator through a first electric circuit (not shown). The metal plate can be in communication with the electronic indicator through a second electric circuit (not shown). The electronic indicator is configured to alert an operator when the metal mesh is penetrated and when the metal plate is contacted by a puncturing needle or other tracheostomy tool.

In an embodiment, the perforations 20 include a series of micro-perforations or small, adjacent holes or slits arranged in a pre-determined line. The perforations 20 can be arranged in a line such that the distal wall portion 12 can be separated along the line upon application of slight pressure. For example, a puncturing needle can be inserted through the perforations 20 and penetrate the metal mesh, thereby causing the distal wall portion 12 to be separated along the perforated line and allowing for subsequent passage of a dilator and tracheostomy tube through the distal wall portion 12. The perforations 20 can facilitate removal of the endotracheal tube 10 from the trachea once the tracheostomy tube is properly positioned.

The metal plate 26 of the posterior wall segment 24 can be aligned with the line of perforations 20, such that the metal plate 26 is positioned directly behind the line of perforations 20 or directly facing the line of perforations 20. In an embodiment, the metal plate 26 can include first and second parallel, opposing sides, third and fourth parallel, opposing sides, and a fifth slanted side extending between and connecting the second and third sides. The metal plate 26 can be puncture-resistant and can be configured to alert the operator if the needle contacts the metal plate 26. The metal plate 26 can be positioned within the posterior wall portion 24 of the endotracheal tube 10 and can function in three ways: (1) providing connectivity for the second circuit; (2) reinforcing the inner wall of the endotracheal tube to protect the esophagus from a needle puncture during the percutaneous tracheostomy; and (3) allowing for visualizing the endotracheal tube by X-ray to aid with the orientation of the tube and rotation around its axis.

In an embodiment, the metal plate 26 can aid in positioning of the endotracheal tube 10. The endotracheal tube can be positioned at an appropriate depth and orientation within the trachea, by visualizing the metal plate 26 with X-ray imaging. The endotracheal tube can be properly positioned within the trachea once the metal plate of the endotracheal tube is positioned between tracheal rings 2 and 3 and the anterior or front wall portion of the endotracheal tube is facing the anterior part of the neck. The appropriate positioning of the endotracheal tube can be determined by visualizing the metal plate of the posterior portion of the tube using a simple X-ray. If the plate appears on the X-ray image as aligned with tracheal rings 2 and 3 and the slanted side of the plate appears on the appropriate side, then a determination can be made that the endotracheal tube is properly positioned.

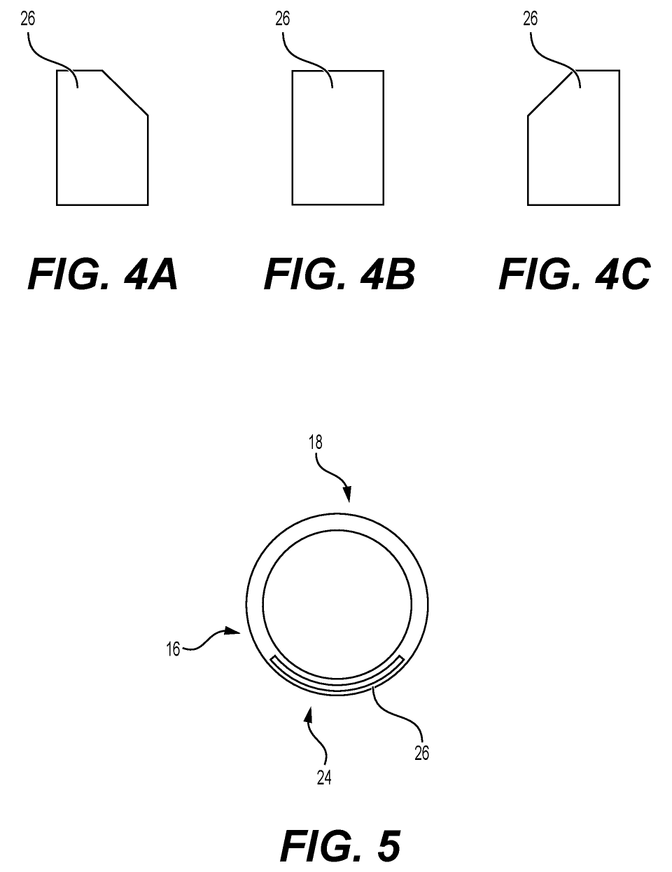

In an embodiment, the positioning of the metal plate, as shown in an X-ray image of the endotracheal tube, can be used to determine appropriate positioning of the endotracheal tube. In an embodiment, if the X-ray image shows the slanted side of the metal plate at the top right of the metal plate, as shown in FIG. 4A, a determination can be made that the endotracheal tube is properly positioned. In an embodiment, if the slanted side of the metal plate cannot be seen in an X-ray image, it can be determined that the endotracheal tube is improperly rotated. In an embodiment, if the slanted side of the metal plate can be seen in the top left of the metal plate in an X-ray image, it can be determined that the endotracheal tube is in a flipped orientation (FIG. 4C).

The metallic mesh 22 in the anterior segment of the distal wall portion 12 is configured to sense the penetration of the needle of the tracheostomy tube. The mesh is electroconductive and connected to a first electrical circuit. When the puncturing needle contacts the metallic mesh, the second electric circuit becomes complete causing an electric signal to be sent to the electronic indicator and triggering an alarm, e.g., a visual or sound alarm. In an embodiment, the mesh is formed from metal strings that can be easily separated by pressure applied from a puncturing needle for inserting the endotracheal tube.

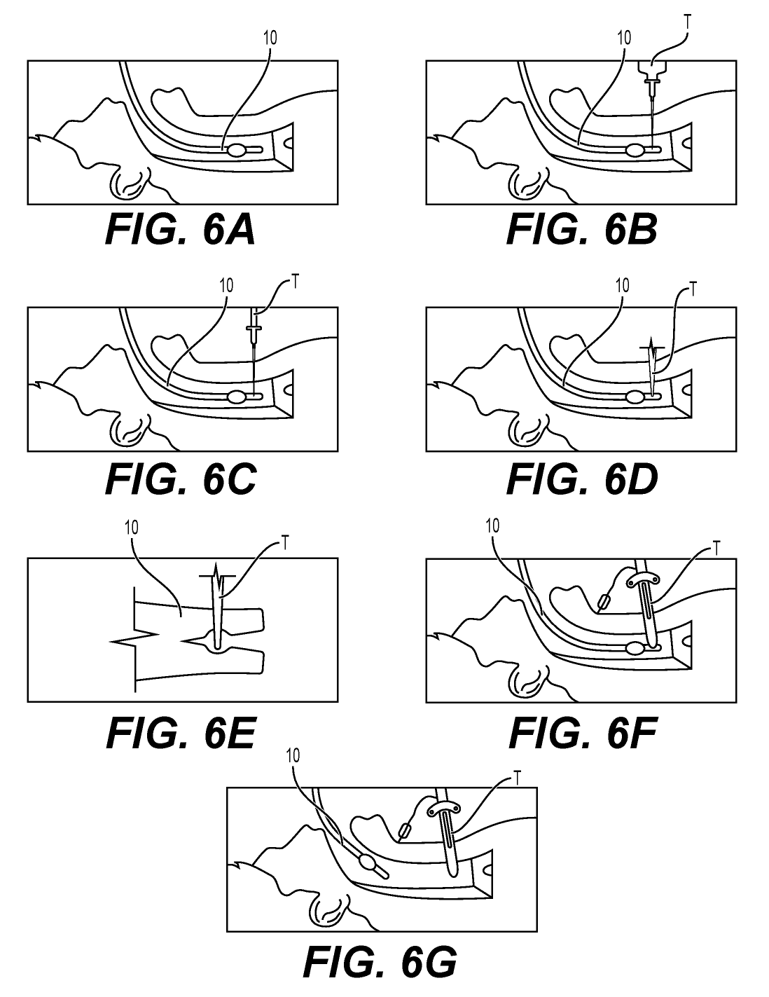

Once the needle punctures and passes through the metal mesh (FIG. 6B), the first electrical circuit becomes complete, thereby activating the first electronic indicator. Activation of the first electronic indicator alerts the operator that the puncturing needle has been properly positioned in the endotracheal tube. If the operator advances the needle slightly further, the needle will contact the metal plate, positioned directly behind the line of perforations in the posterior wall segment of the endotracheal tube, leading to a complete connection of the second circuit and activation of the electronic indicator.

In an embodiment, the first and second electronic indicator can be selected from the group consisting of a light in a twin lightbox, a notification alert displayed on a monitor M, and a sound alert (e.g., beeping or voice message).

As described herein, the endotracheal tube 10 is configured to receive a tracheostomy tube. Separation of the distal wall portion of the endotracheal tube along the perforations can also allow the endotracheal tube to be removed from the patient while simultaneously inserting the tracheostomy tube. Unlike current practice, which requires pulling back the endotracheal tube prior to starting the percutaneous tracheostomy procedure, the present endotracheal tube remains in place during tracheostomy insertion. The endotracheal tube provides guidance during the insertion, and protection of the tracheal wall, as well as eliminating the need for a second operator and a bronchoscope. Without the perforations, it would not be possible to remove the endotracheal tube while inserting the tracheostomy tube and ensuring accurate positioning.

The endotracheal tube of the present teachings also obviates the need for bronchoscopy because X-ray imaging can be used to confirm proper placement of the endotracheal tube, such that the metal plate of the endotracheal tube is aligned between the 2nd and 3rd tracheal rings. As X-ray imaging is also used to confirm conventional percutaneous tracheostomy procedures, utilization of X-ray to confirm proper placement of the endotracheal tube of the present teachings does not incur any additional costs.

The endotracheal tube of the present teachings can be administered in a conventional manner and confirmation of proper placement of the endotracheal tube can be provided by X-ray imaging of the metal plate as discussed above. Once the endotracheal tube is properly positioned, a tracheostomy tube can be positioned within the trachea using the endotracheal tube as a guide.

An exemplary method for placement of the tracheostomy tube is depicted in FIGS. 6A-6G. Initially, a skin incision is made in a conventional manner. Unlike conventional practice which uses bronchoscopy for guidance during the needle puncture, the first and second electrical circuits of the present endotracheal tube provide confirmation of proper needle placement (FIGS. 6A-6B). Once the needle is properly positioned, the syringe attached to the puncturing needle is removed and a guiding wire is advanced into the needle (FIG. 6C). Unlike conventional practice, the guidewire can be advanced through the needle to the already-in-place endotracheal tube, rather than directly in the trachea. After inserting the guiding wire, the needle can be removed and first-stage dilation can be performed by, e.g., using a small dilator. Then, the dilator can be removed and the guidewire protector can be inserted. Then, the second-stage dilator (large dilator) e.g, Rhino™ dilator, can be placed over the guidewire and the guidewire protector (FIG. 6D). Similar to the guiding wire, the dilators can be inserted into the endotracheal tube and not directly in the trachea. The dilator can be advanced while applying pressure to the distal end of the endotracheal tube to facilitate an easy and controlled separation of the distal wall portion along the perforated line (FIG. 6E). Afterwards, the dilator can be removed, and the tracheostomy tube can be inserted over the guiding wire (FIG. 6F) while the endotracheal tube is removed simultaneously (FIG. 6G). Similar to the current practice, an X-ray can be performed to confirm the accurate placement of the tracheostomy tube at the end of the procedure.

It is to be understood that the endotracheal tube is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An endotracheal tube, comprising:
a tubular wall having an open proximal end, an opposing open distal end, and a distal wall portion spaced from the open proximal end and extending to the open distal end;
a plurality of perforations defined through an anterior wall segment of the distal wall portion;
a metal mesh formed along the anterior wall segment, the metal mesh configured to be in communication with an electronic indicator; and
a metal plate formed along a posterior wall segment of the distal wall portion, the metal plate configured to be in communication with the electronic indicator.

2. The endotracheal tube according to claim 1, wherein the metal plate includes first and second parallel, opposing sides, third and fourth parallel, opposing sides, and a fifth slanted side extending between and connecting the second and third sides.

3. The endotracheal tube as recited in claim 1, wherein the metal mesh is in communication with the electronic indicator through a first electric circuit and the metal plate is in communication with the electronic indicator through a second electric circuit.

4. The endotracheal tube according to claim 1, wherein the plurality of perforations comprise adjacent holes arranged in a line.

5. The endotracheal tube according to claim 4, wherein the metal plate is directly facing the plurality of perforations of the anterior wall segment.

6. An endotracheal tube, comprising:
a tubular wall having an open proximal end, an opposing open distal end, and a distal wall portion spaced from the open proximal end and extending to the open distal end;
a plurality of perforations defined through an anterior wall segment of the distal wall portion;
a metal mesh formed along the anterior wall segment, the metal mesh configured to be in communication with an electronic indicator; and
a metal plate formed along a posterior wall segment of the distal wall portion, the metal plate including first and second parallel, opposing sides, third and fourth parallel, opposing sides, and a fifth slanted side extending between and connecting the second and third sides, and the metal plate configured to be in communication with the electronic indicator.

7. The endotracheal tube as recited in claim 6, wherein the metal mesh is in communication with the electronic indicator through a first electric circuit and the metal plate is in communication with the electronic indicator through a second electric circuit.

8. The endotracheal tube as recited in claim 6, wherein the plurality of perforations comprise adjacent holes arranged in a line.

9. The endotracheal tube as recited in claim 6, wherein the metal plate is directly facing the plurality of perforations of the anterior wall segment.

10. An endotracheal tube, comprising:
a tubular wall having an open proximal end, an opposing open distal end, and a distal wall portion spaced from the open proximal end and extending to the open distal end;
a plurality of perforations defined through an anterior wall segment of the distal wall portion;
a metal mesh formed along the anterior wall segment, the metal mesh configured to be in communication with an electronic indicator; and
a metal plate formed along a posterior wall segment of the distal wall portion, the metal plate configured to be in communication with the electronic indicator,
wherein the metal mesh is in communication with the electronic indicator through a first electric circuit and the metal plate is in communication with the electronic indicator through a second electric circuit.

11. The endotracheal tube as recited in claim 10, wherein the metal plate includes first and second parallel, opposing sides, third and fourth parallel, opposing sides, and a fifth slanted side extending between and connecting the second and third sides.

12. The endotracheal tube as recited in claim 10, wherein the plurality of perforations comprise adjacent holes arranged in a line.

13. The endotracheal tube as recited in claim 12, wherein the metal plate is directly facing the plurality of perforations of the anterior wall segment.
